(19) United States
(12) Patent Application Publication
(10) Pub. No.: US 2009/0304766 A1
(43) Pub. Date: Dec. 10, 2009

(54) LOCALIZED DELIVERY OF DRUG COMBINATIONS

(76) Inventor: Lawrence Mayer, North Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 12/095,494

(22) PCT Filed: Nov. 30, 2006
(86) PCT No.: PCT/US06/46159

§ 371 Date: Oct. 3, 2008

Related U.S. Application Data
(60) Provisional application No. 60/740,833, filed on Nov. 30, 2005.

(51) Int. Cl. A61F 2/04

(52) U.S. Cl. 424/423; 424/422

(57) ABSTRACT

Implants comprising controlled delivery matrices, associated with, or including medical devices, having stably and releasably associated therewith predetermined non-antagonistic combinations of two or more therapeutic agents provide control of the ratio of these agents at a localized site. Methods of identifying such combinations are also disclosed.

### RELATED APPLICATION

This application claims benefit of U.S. application Ser. No. 60/740,833 filed 30 Nov. 2005, which is incorporated herein by reference in its entirety.

### TECHNICAL FIELD

The invention relates to compositions and methods for localized delivery of synergistic or additive combinations of therapeutic agents. More particularly, the invention concerns delivery systems which ensure the maintenance of synergistic or additive ratios when the agents are delivered locally to a target.

### BACKGROUND ART

Implantation of medical devices such as stents, wafers, pastes or reservoirs has been used to deliver a therapeutic agent locally for a number of medical applications including surgical adhesions, treatment of inflammatory arthritis, treatment of scars and keloids, the treatment of vascular disease, and the prevention of cartilage loss. For example, paclitaxel-eluting stents have been widely tested in cardiac patients to reduce the recurrence of restenosis among other complications of heart surgery. Although shown to be effective at delivering a single pharmaceutical agent, local delivery of multiple agents has yet to be contemplated.

Because the progression of many life-threatening diseases such as cancer, AIDS, immune and cardiovascular disorders is influenced by multiple molecular mechanisms, achieving cures with a single agent has been met with limited success. Thus, for many systemically treated diseases, combinations of agents have often been used to combat disease, particularly in the treatment of cancers. It appears that there is a strong correlation between the number of agents administered and cure rates for cancers such as acute lymphocytic leukemia. (Frei, et al., Clin. Cancer Res. (1998) 4:2027-2037). Clinical trials utilizing combinations of doxorubicin, cyclophosphamide, vincristine, methotrexate with leucovorin rescue and cytarabine (ACOMLA) or cyclophosphamide, doxorubicin, vincristine, prednisone and bleomycin (CHOP-b) have been successfully used to treat histiocytic lymphoma (Todd, et al., J. Clin. Oncol. (1984) 2:986-993).

The effects of combinations of drugs are enhanced when the ratio in which they are supplied provides a synergistic effect. Synergistic combinations of free agents have also been shown to reduce toxicity due to lower dose requirements, to increase cancer cure rates (Barriere, et al., Pharmacotherapy (1992) 12:397-402; Schimpff, Support Care Cancer (1993) 1:5-18), and to reduce the spread of multi-resistant strains of microorganisms (Shlaes, et al., Clin. Infect. Dis. (1993) 17:S527-S536). By choosing agents with different mechanisms of action, multiple sites in biochemical pathways can be attacked thus resulting in synergy (Shah and Schwartz, Clin. Cancer Res. (2001) 7:2168-2181). Combinations such as L-canavanine and 5-fluorouracil (5-FU) have been reported to exhibit greater antineoplastic activity in rat colon tumor models than the combined effects of either drug alone (Swaffar, et al., Anti-Cancer Drugs (1995) 6:586-593). Cisplatin and etoposide display synergy in combating the growth of a human small-cell lung cancer cell line, SBC-3 (Kanzawa, et al., Int. J. Cancer (1997) 71(3):311-319).

In the foregoing studies, the importance of the ratio of the components for synergy was recognized. For example, 5-fluorouracil and L-canavanine were found to be synergistic at a mole ratio of 1:1, but antagonistic at a ratio of 5:1; cisplatin and carboplatin showed a synergistic effect at an area under the curve (AUC) ratio of 13:1 but an antagonistic effect at 19:5.

Other drug combinations have been shown to display synergistic interactions although the dependency of the interaction on the combination ratio was not described. This list is quite extensive and is composed mainly of reports of in vitro cultures, although occasionally in vivo studies are included. In addition to the multiplicity of reports, numerous combinations have been shown to be efficacious in the clinic.

Despite the advantages associated with the use of systemically-administered synergistic drug combinations, there are various drawbacks that limit their therapeutic use. For instance, synergy often depends on various factors such as the duration of drug exposure and the sequence of administration (Bonner and Kozelsky, Cancer Chemother. Pharmacol. (1990) 39:109-112). Studies using ethyl deshydroxy-sparsomycin in combination with cisplatin show that synergy is influenced by the combination ratios, the duration of treatment and the sequence of treatment (Hofs, et al., Anticancer Drugs (1994) 5:35-42).

It is known that in order for synergy to be exhibited by a combination of agents, these agents must be present in defined ratios as outlined in PCT publication WO 03/028696 incorporated herein by reference. The same combination of drugs may be antagonistic at some ratios, synergistic at others, and additive at still others. It is desirable to avoid antagonistic effects, so that the drugs are at least additive. Furthermore, whether a particular ratio is synergistic, additive, or antagonistic at a target site is concentration dependent. Thus it is desirable to better control both the concentration and ratio of the drugs which reaches the target. The problem is solved for systemic administration as described in the above-cited PCT publication.

The problem of controlling these ratios and thus maintaining synergy or additivity is solved for local delivery by the recognition that when therapeutic agents delivered locally in a controlled manner, e.g., associated with medical devices, such as wafers, pastes, stents, reservoirs or films, the devices themselves (including drug-carrying matrices incorporated in said devices) help control the drug release rates and thus drugs associated with such devices will be released in a similar manner. Therefore, in contrast to free drug cocktails which provide no control over the release rate of the drugs, combinations of agents associated with delivery devices at predefined ratios will be locally delivered to the target cells and or tissues at the desired ratio(s).

In vitro analysis of drug interactions can be used to identify non-antagonistic ratios of drugs which can then be stably associated with one or more release matrices or medical devices for local drug delivery. In one embodiment, the drugs are first formulated within a matrix (such as a polymer film, micelle or hydrogel) which is supplied in or with a device (e.g., coated on or impregnated within the device) from which the drugs are released at a comparable rate. Since the drugs are associated with the device at the desired non-antagonistic ratio and are released locally at a comparable rate, the target cells/tissues surrounding the device are exposed to the agents at the desired non-antagonistic ratio thereby maximizing their combined therapeutic effects.

The present invention relates to compositions and methods which allow for the controlled, local delivery of non-antagonistic combinations of two or more therapeutic agents by identifying and ‘fixing’ these combinations in appropriate medical drug eluting devices or compositions.

### DISCLOSURE OF THE INVENTION

The compositions and methods of the present invention provide combination drug therapies that ensure a non-antagonistic drug combination will be locally delivered to the target site at a desired non-antagonistic ratio by stably associating the drugs at said ratio into controlled release compositions or medical devices such that each drug is released at a comparable rate. Therefore, the drugs are released from and exposed to local cells/tissues at the desired ratio. Identification of ratios of therapeutic agents which provide non-antagonistic effects over a range of concentrations is preferably achieved by selecting combinations of therapeutic agents which are shown to be non-antagonistic in vitro. When significant differences exist between the cytotoxicity curves of the individual agents relative to each other in vitro and the relative biological potencies of these agents in vivo, methods can be used to correct for these discrepancies and thus identify a combination of the agents which will provide maximum efficacy in vivo.

Thus, in one aspect, the invention provides an implant that comprises a controlled delivery matrix, optionally contained in a device or which is itself a device, for local administration comprising two or more agents included in or on the matrix at a ratio that is synergistic or additive. In one embodiment, a medical device is prepared by a process comprising first, formulating the agents in a matrix (such as a polymer film, micelle or hydrogel) at these ratios and then stably associating the drugs/matrix with the medical device. Alternatively, the medical device may first be provided with the matrix, and then the desired ratio of drugs introduced, or one of the drugs may be placed in the matrix before it is associated with a medical device and the second drug added subsequent to this step. In some cases, the agents may, themselves, be coated on or included in the device. Also, some matrices are sufficiently able to maintain an intact composition so as to behave as implants themselves.

In another aspect, the invention provides an implant which is a controlled delivery matrix wherein the first and/or second agent, in a non-antagonistic ratio, are included directly in a medical device. In this embodiment, the controlled delivery matrix is itself a medical device. The agents may be coated on the surface of the device, or may be impregnated within it or one of the agents may be coated and the other impregnated in the device. Alternatively, one of the agents may be contained independently in an associated matrix and the other directly included. The non-antagonistic ratio of the agents in this embodiment obey the same parameters as those wherein the agents are included in a matrix that is not itself a medical device.

The non-antagonistic ratio of the agents is determined by assessing the biological activity or effects of the agents on relevant cell culture or cell-free systems over a range of concentrations and, in one embodiment, applying an algorithm to determine a “combination index,” (CI). As further described in PCT application WO 03/028696 (which is incorporated herein by reference), using recognized algorithms, a combination index can be calculated at each concentration level. Ratios are selected where the CI represents synergy or additivity over a range of concentrations. Similarly, in vivo model systems may be used to determine suitable non-antagonistic ratios. In one embodiment, the agents are antitumor agents. Any method which results in determination of a ratio of agents which maintains a non-antagonistic effect over a desired range of concentrations may be used.

The invention thus, in one embodiment, relates to a matrix composition optionally associated with a medical device, said matrix having stably but releasably associated therewith at least a first therapeutic agent and a second therapeutic agent in a mole ratio of the first agent to the second agent which exhibits a non-antagonistic biologic effect to relevant cells in culture or cell-free system over at least 5% of such concentration range where greater than 1% of the cells are affected (Fraction affected (fₐ)>0.01) or to a medical device, having coated thereon or encapsulated therein at least a first therapeutic agent and a second therapeutic agent in a mole ratio of the first agent to the second agent which exhibits a non-antagonistic biological effect to relevant cells, for example, to the extent described above. The agents may be antineoplastic agents or agents that affect endogenous chronic disease states such as inflammation, or they may be antibiotics or antiviral agents. By “biological effect”, applicants refer to cytotoxic or cytostatic effects, or other events such as inhibition of endotoxin- or cytokine-mediated activation of macrophage, inhibition of degranulation, superoxide generation, migration of leukocytes, inhibition of proliferation of endothelial or smooth muscles cells, among other endpoints of toxicity or activity. By “relevant” cells, applicants refer to at least one cell culture or cell line which is appropriate for testing the desired biological effect. For example, if the agent is an antineoplastic agent, a “relevant” cell would be a cell line identified by the Developmental Therapeutics Program (DTP) of the National Cancer Institute (NCI)/National Institutes of Health (NIH) as useful in their anticancer drug discovery program. Currently the DTP screen utilizes 60 different human tumor cell lines. The desired activity on at least one of such cell lines would need to be demonstrated.

In another aspect, the invention is directed to a method to deliver a synergistic or additive ratio of two or more therapeutic agents to a desired target by providing the implants, i.e., the devices or controlled delivery matrices of the invention.

### BRIEF DESCRIPTION OF THE DRAWINGS

FIG. 1 is a diagram outlining the method of the invention for determining an appropriate ratio of therapeutic agents to include in formulations.

FIGS. 2A-2E show various methods for presenting in vitro combination and synergy data.

### MODES OF CARRYING OUT THE INVENTION

The invention involves formulating combination drug therapies which allow for local delivery of non-antagonistic ratios of two or more therapeutic agents by stably associating the therapeutic agents into implants, used for local drug delivery, at predefined non-antagonistic ratios of the two or more therapeutic agents.

One method of the invention involves determining a ratio of therapeutic agents which is non-antagonistic over a desired concentration range in vitro and supplying this non-antagonistic ratio in a manner that will ensure that the ratio is delivered to a local site of desired activity. This method is described in detail in the above-cited PCT publication WO 03/028696. Briefly, the synergistic or additive ratio is determined by applying standard analytical tools to the results obtained when at least one ratio of two or more therapeutic agents is tested in vitro over a range of concentrations against relevant cell cultures or cell-free systems. By way of illustration, individual agents and various combinations thereof are tested for their biological effect on cell culture or a cell-free system, for example causing cell death or inhibiting cell growth, at various concentration levels. The concentration levels of the preset ratios are plotted against the percentage cell survival to obtain a correlation which can be manipulated by known and established mathematical techniques to calculate a “combination index” (CI). The mathematics are such that a CI of 1 (i.e., 0.9-1.1) describes an additive effect of the drugs; a CI>1 (i.e., >1.1) represents an antagonist effect; and a CI of <1 (i.e., <0.9) represents a synergistic effect.

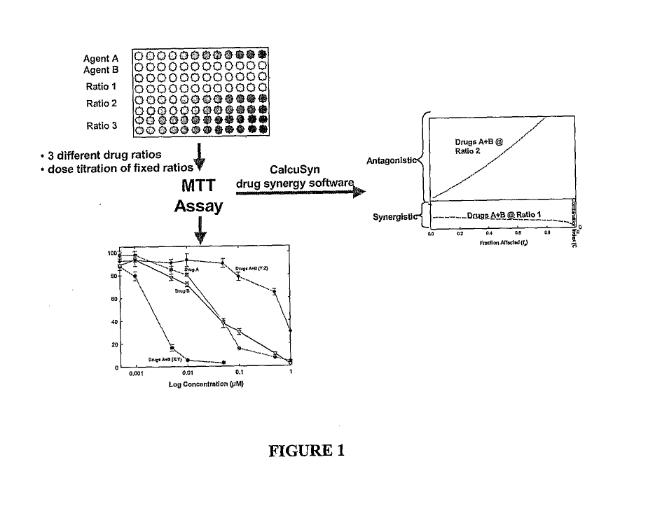

One general approach is shown in FIG. 1. As shown, agents A and B are tested individually and together at two different ratios for their ability to cause cell death or cell stasis as assessed by the MTT assay described below. Initially, correlations between the concentration of drugs A, B, and the two different combination ratios (Y:Z and X:Y) are plotted against cytotoxicity, calculated as a percentage based on the survival of untreated control cells. As expected, there is a dose-dependent effect on cell survival both for the individual drugs and for the combinations. Once this correlation has been established, the cell survival or fraction affected (fₐ) can be used as a surrogate for concentration in calculating the CI.

The results of the CI calculation are also shown in FIG. 1; this index is calculated as a function of the fraction of cells affected according to the procedure of Chou and Talalay, Advance Enz. Regul. (1985) 22:27-55. In this hypothetical situation, the first ratio (X:Y) of drugs A plus B is non-antagonistic at all concentrations while the combination in the second ratio (Y:Z) is antagonistic. Thus, it is possible to provide a ratio of drugs A plus B (ratio 1) which will be non-antagonistic regardless of concentration over a wide range. It is this ratio that is preferable to include in the compositions of the invention.

While the determination in vitro of non-antagonistic ratios has been illustrated for a combination of only two drugs, application of the same techniques to combinations of three or more drugs provides a CI value over the concentration range in a similar manner. In addition, account may be taken of differences in drug activity in vitro as opposed to in vivo. One drug in the combination may exhibit a different ratio of in vivo vs. in vitro activity as compared to the ratio exhibited by the other. If so, this is corrected for in the formulation. Another method of determining suitable non-antagonistic ratios is by taking account of maximal therapeutic dosages.

Identification of non-antagonistic ratios of particular therapeutic agents may be achievable through additional means, such as published literature and/or past documentation. In addition, while generally less convenient, in vivo models may be used to determine biological effects and combinations that are at least additive.

By “therapeutic agent” is meant any agent that has a beneficial effect on the subject. The beneficial effect may be prophylactic—i.e., preventative, or may be ancillary to an intended effect, such as affecting mechanisms for drug resistance. Any agent with a desired biological effect on a subject is included in the definition of “therapeutic agent.”

In one embodiment, the ratio is maintained in the pharmaceutical composition by first stably associating the agents at the predetermined ratio in a polymer film, micelle, hydrogel, or other matrix which assures that the non-antagonistic ratio will be maintained and optionally, coating or impregnating a medical device with the drug/matrix formulation which is then implanted in or administered to the patient. In an alternative approach, a matrix may be associated with a device and then provided with the desired ratio of drugs. If the matrix is of sufficient integrity and hardness, it itself may behave as an implantable medical device. In another embodiment, the device is provided directly with the drugs in stable association with the device, or one drug may be provided directly to the device and the other contained in a matrix, which may optionally be associated with the device. Various permutations of means of preparation and association of the two or more active agents with implantable devices or matrices are included within the scope of the invention. The matrices themselves or medical devices that provide the desired ratio of therapeutic agents stably associated therewith are implanted to provide local administration.

While it is preferred to “coencapsulate” the agents so that both are contained in the same matrix and/or device, this is not necessary. Since drug-eluting matrices and/or devices can display similar drug release rates, the active substances experience coordinated release from the formulation even if encapsulated in separate devices and/or matrices so long as the drug release rates from the devices are coordinated at the implant site.

A “controlled delivery matrix” is a composition, optionally in particulate or a solid form, that can stably associate with a therapeutic agent but control its release. It can be used alone if constructed as a macro-article or can be coated onto or formulated within a medical device, or it may, itself, be a medical device. Matrices to accommodate the combination of therapeutic agents may include, but are not limited to, polymer films, micelles, hydrogels, liposomes or other lipidic or polymeric pastes. The matrices including devices are chosen such that they result in similar release rates of each therapeutic agent being administered such that the desired ratio of agents that is maintained at the local target site.

Terms and phrases such as “encapsulated” or “stably associated” or “stably and releasably associate” or “coated in a stable and releasable manner”, mean that the agents are retained in the matrix and/or device in a controlled manner so that the ratio of the combined agents is sufficiently maintained and released as desired on exposure to the disease site. Thus, for example, it is not necessary for the matrix to surround the therapeutic agent or agents as long as the agent or agents is/are stably associated with the matrix when coated onto or in the medical device. “Stably associated with” and “encapsulated in” or “encapsulated with” or “impregnated in or with” or “co-encapsulated in or with” or “formulated in or with”, etc, are intended to be synonymous terms. They are used interchangeably in this specification. The stable association may be effected by a variety of means, including covalent bonding to the matrix or device, preferably with a cleavable linkage, noncovalent bonding, and trapping the agent in the interior or bulk of a matrix and the like. The association must be sufficiently stable so that the agents remain associated with the matrix at a non-antagonistic ratio until it is delivered to the local target site in the treated subject, but must release the drugs in a controlled rate. Similarly, the association of the drug-carrying matrices with a medical device must be sufficiently stable so that said matrices remain associated with the medical device so as to allow for implantation/absorption of the device.

The required “stable and releasable association” simply means that a non-antagonistic ratio will be found in any appropriate sample, such as blood or other fluid or tissue associated with the desired site over a time period effective for treatment which may be over a period of 1 hour, 12 hours, 24 hours or 48 hours. The ratio is most conveniently measured in the blood or serum as an indication of the ratio at the site from which the blood or serum is drawn.

Thus, an implant that includes a “controlled delivery matrix” will include any implantable material, or combination of implantable materials. The “controlled delivery matrix” will have two or more agents “stably and releasably” associated with it to effect localized delivery of a non-antagonistic ratio of these agents over a suitable time period. The non-antagonistic ratio can be measured in the blood or serum as well as in tissues associated with the controlled delivery matrix.

Preferably, the compositions of the invention are used to locally deliver combinations of encapsulated therapeutic agents that are synergistic.

### Preparation of Matrices and Devices Containing Encapsulated Drugs

In one embodiment, when appropriate non-antagonistic ratios of the agents have been determined, the agents at the desired ratio are stably associated with a suitable matrix composition wherein one or more matrix encapsulates two or more agents. Not all the matrices in the composition need be identical, but they should result in similar release rates of the associated agents.

As set forth above, the therapeutic agents are stably associated with the matrices, include those matrices that are medical devices. The therapeutic agents may be formulated together with the same matrix or may be associated with different compositions in the same device or implant. If different compositions are used, the drug release rates from the compositions are preferably “coordinated.” By compositions with “coordinated” release rates is meant that the compositions assure maintenance of the ratio of the therapeutic agents administered at a non-antagonistic ratio; even if they are delivered to target tissues in other than the same composition.

Stable association with the matrix may be achieved by interaction of the agent with the outer layer or layers of the matrix or entrapment of an agent within the matrix, equilibrium being achieved between different portions of the matrix, and by covalent or non-covalent interaction. For example, encapsulation of an agent in liposomes can be by association of the agent by interaction with the bilayer of the liposomes through covalent or non-covalent interaction with the lipid components or entrapment in the aqueous interior of the liposome, or in equilibrium between the internal aqueous phase and the bilayer prior to associating the liposomes with a medical device. For polymer-based matrices, encapsulation can refer to covalent linkage of an agent to a linear or non-linear polymer. Further, non-limiting examples include the dispersion of agent throughout a polymer matrix, or the concentration of drug in the core or dispersed throughout a nanocapsule, a polymer micelle or a polymer-lipid hybrid system. “Loading” refers to the act of encapsulating one or more agents into a matrix.

Encapsulation of the desired combination can be achieved either through encapsulation in separate matrices or within the same matrix and will be optimized for individual drugs. The matrix formulation will be chosen as that which optimally loads and/or retains both drugs in the combination. By altering the matrix composition, release rates of encapsulated drugs can be matched to allow non-antagonistic ratios of the drugs to be delivered to the target site.

Techniques for encapsulation are dependent on the nature of the matrix and the nature of any device that is directly associated with an agent.

Once the combination of therapeutic agents have been formulated in a suitable composition at a non-antagonistic ratio, the drug-containing complex may then be stably associated with a device used for incorporation into or administration to a patient and thus delivery of the combination of therapeutic agents. The drug-containing complex may be coated on, impregnated within or otherwise incorporated into the device. For example, polymer films or micelles stably associated with two or more drugs are coated onto a stent which can then be implanted into the arteries of a patient's heart. The drug-eluting stent releases the combination of therapeutic agents over time to treat restenosis or other complications due to heart surgery.

Alternatively, the medical device may be prepared with the drug-containing composition before its association with the combination of therapeutic agents and the non-antagonistic combination of agents may be subsequently associated with the composition.

Devices to be used in the invention include, but are not limited to, stents, wafers, pastes, films, reservoirs, tablets and the like.

### Therapeutic Uses of Medical Device Compositions Encapsulating Multiple Agents

These ratio-specific device compositions may be used to treat a variety of diseases. Thus, suitable subjects for treatment according to the methods and compositions of the invention include humans, mammals such as livestock or domestic animals, domesticated avian subjects such as chickens and ducks, and laboratory animals for research use.

As mentioned above, the ratio-specific matrices/devices of the present invention may be implanted in or on warm-blooded animals, including humans as well as domestic avian species. For treatment of human ailments, a qualified physician will determine how the patient-specific compositions of the present invention should be implanted/administered with respect to dose, schedule and route of administration. Such applications may also utilize dose escalation should agents encapsulated in device compositions of the present invention exhibit reduced toxicity to healthy tissues of the subject.

The preferred embodiments herein described are not intended to be exhaustive or to limit the scope of the invention to the precise forms disclosed. They are chosen and described to best explain the principles of the invention and its application and practical use to allow others skilled in the art to comprehend its teachings.

The following examples are offered to illustrate but not to limit the invention.

### EXAMPLES

### Example 1

### Multiple Representation of Dose-Effect Analysis

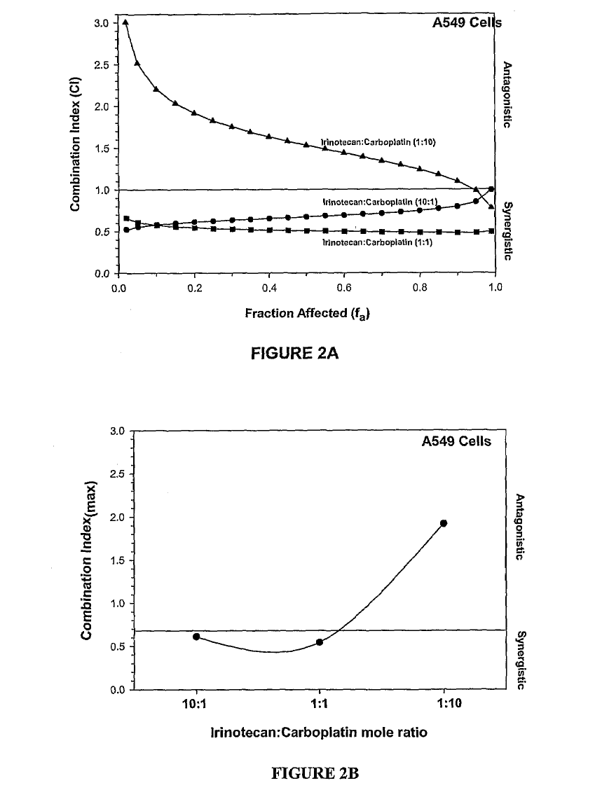

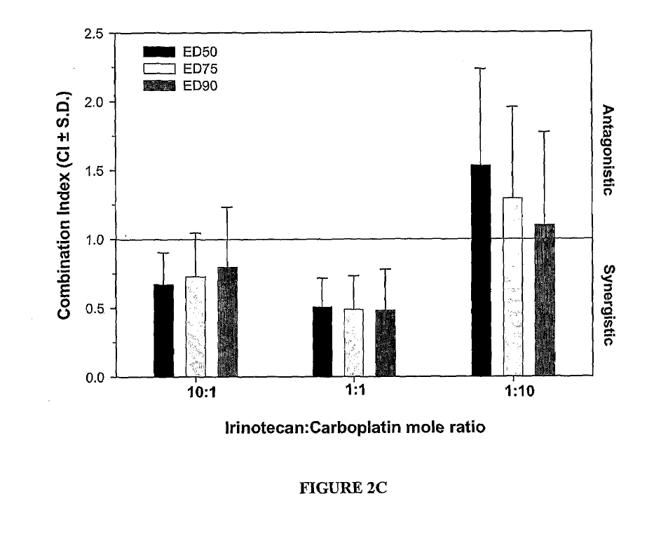

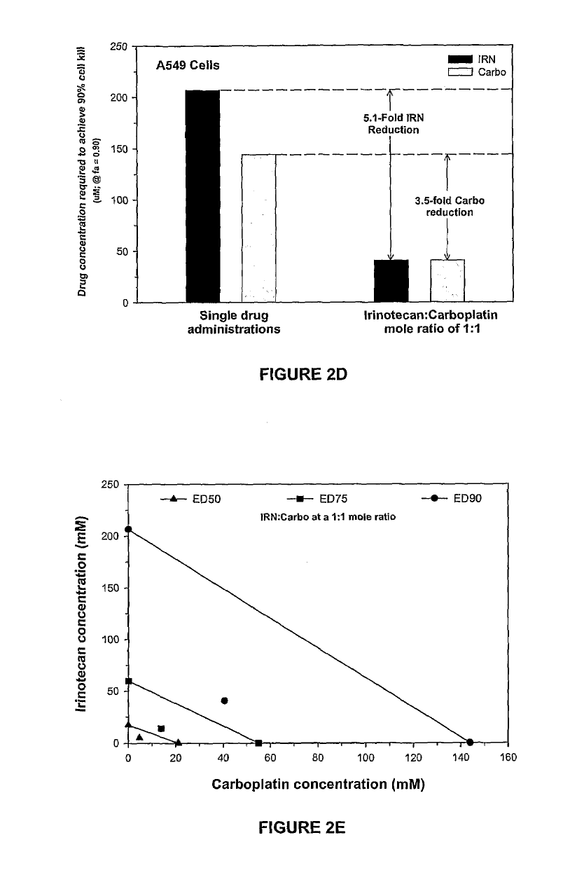

Quantitative analysis of the relationship between an amount (dose or concentration) of drug and its biological effect as well as the joint effect of drug combinations can be measured and reported in a number of ways. FIGS. 2A-2E illustrate 5 such methods using, as an example, a combination of irinotecan and carboplatin.

Based on Chou and Talalay's theory of dose-effect analysis, a “median-effect equation” has been used to calculate a number of biochemical equations that are extensively used in the art. Derivations of this equation have given rise to higher order equations such as those used to calculate Combination Index (CI). As mentioned previously, CI can be used to determine if combinations of more than one drug and various ratios of each combination are antagonistic, additive or synergistic. CI plots are typically illustrated with CI representing the y-axis versus the proportion of cells affected, or fraction affected (fₐ), on the x-axis. FIG. 2A demonstrates that a 1:10 mole ratio of irinotecan/carboplatin is antagonistic (CI>1.1), while 1:1 and 10:1 have a synergistic effect (CI<0.9).

The present applicants have also designed an alternative method of representing the dependency of CI on the drug ratios used. The maximum CI value is plotted against each ratio to better illustrate trends in ratio-specific effects for a particular combination as seen in FIG. 2B. The CI maximum is the CI value taken at a single fₐ value (between 0.2 and 0.8) where the greatest difference in CI values for the drugs at different ratios was observed.

Because the concentrations of drugs used for an individual ratio play a role in determining the effect (i.e., synergism or antagonism), it can also be important to measure the CI at various concentrations. These concentrations, also referred to as “Effective Doses” (ED) by Chou-Talalay, are the concentration of drug required to affect a designated percent of the cells in an in vitro assay, i.e., ED₅₀ is the concentration of drug required to affect 50% of the cells relative to a control or untreated cell population. As shown in FIG. 2C, trends in concentration-effect are readily distinguished between the various ratios. The error bars shown represent one standard deviation around the mean and is determined directly through the CalcuSyn program.

A synergistic interaction between two or more drugs has the benefit that it can lower the amount of each drug required in order to result in a positive effect, otherwise known as “dose-reduction.” Chou and Talalay's “dose-reduction index” (DRI) is a measure of how much the dose of each drug in a synergistic combination may be reduced at a given effect level compared with the doses for each drug alone. DRI has been important in clinical situations, where dose-reduction leads to reduced toxicity for the host while maintaining therapeutic efficacy. The plot in FIG. 2D shows that the concentrations of irinotecan and carboplatin required to achieve a 90% cell kill on their own is significantly higher than their individual concentrations required when they are combined at a non-antagonistic ratio.

Furthermore the aforementioned data can be represented in a classical isobologram (FIG. 2E). Isobolograms have the benefit that they can be generated at different ED values; however, they become more difficult to read as more effect levels are selected for interpretation. For this reason, the data in the examples below are generally presented in accordance with the types of plots shown in FIGS. 2A and 2B.

1. An implant comprising a controlled delivery matrix which matrix has stably and releasably associated therewith at least a first therapeutic agent and a second therapeutic agent, such that when said implant is implanted in an in vivo location said agents are delivered to the location in a non-antagonistic ratio.

2. The implant of claim 1, which is a paste or tablet.

3. The implant of claim 1, wherein the controlled delivery matrix is included in a medical device.

4. The implant of claim 3, wherein the medical device is a stent, wafer, or reservoir.

5. The implant of claim 1, which comprises a medical device that includes, in stable and releasable association, at least a first therapeutic agent and a second therapeutic agent, such that when said device is implanted in an in vivo location said agents are delivered to the location in a non-antagonistic ratio.

6. The implant of claim 5, wherein the agents are coated on said device, or
wherein the agents are impregnated in the device, or
wherein the first agent is coated on and the second agent is impregnated in the device.

7. A method to deliver a non-antagonistic ratio of therapeutic agents locally to a subject in need of such delivery, which method comprises providing said subject with the implant of claim 1.

8. A method to prepare the implant of claim 1, which comprises stably and releasably associating with said controlled delivery matrix at least a first therapeutic agent and a second therapeutic agent such that when said implant is implanted in an in vivo location said agents are delivered to the location in a non-antagonistic ratio.

9. The method of claim 8 wherein said implant is or includes a medical device.
